(19) United States
(12) Patent Application Publication
(10) Pub. No.: US 2024/0351021 A1
(43) Pub. Date: Oct. 24, 2024

(54) APPARATUS FOR RAPID DETECTION OF BACTERIA IN FLUIDS

(71) Applicant: The Feinstein Institutes for Medical Research, Manhasset, NY (US)

(72) Inventors: Timir Datta-Chaudhuri, Great Neck, NY (US); Jonathan Robert Danoff, Great Neck, NY (US); Todd Goldstein, Manhasset, NY (US); Stanley E. Asnis, Manhasset, NY (US)

(21) Appl. No.: 18/643,978

(22) Filed: Apr. 23, 2024

Related U.S. Application Data
(60) Provisional application No. 63/461,479, filed on Apr. 24, 2023.

(51) Int. Cl. B01L 3/00

(52) U.S. Cl. CPC B01L 3/502; B01L 2200/025; B01L 2200/026; B01L 2200/0647; B01L 2200/16; B01L 2300/0663; B01L 2300/0681; B01L 2300/16; B01L 2400/043

(57) ABSTRACT

A method and apparatus detects and optionally determines the amounts of bacteria in a fluid sample that may include non-bacterial cells, such as somatic cells. The device has a processing chamber for receiving the sample. The processing chamber includes a somatic cell lysis agent that preferentially disrupts somatic cells to release their contents including somatic cell-derived ATP. The somatic lysis agent is selected to leave bacterial cells intact. The processing chamber also holds a plurality of beads that have an ATP-reactive enzyme, such as ATPase, bound to their surfaces. The somatic cell-derived ATP and endogenous ATP in the sample are eliminated by reaction with the ATP-reactive enzyme. Once the reaction reaches an end point, the sample is transferred to a detection chamber while the beads and the bound ATP-reactive enzymes are retained in the processing chamber. The detection chamber holds a bacterial cell lysis agent that disrupts bacteria that might be present in the sample to release bacterial cell-derived ATP. A light-emitting reagent that reacts with ATP, such as luciferin/luciferase, is provided in the detection chamber. A light sensor monitors the detection chamber to determine if light is emitted, indicating the presence and optionally the amount of bacteria in the fluid sample.

### RELATED APPLICATIONS

This application claims priority under 35 U.S.C. § 119 to U.S. Provisional Patent Application No. 63/461,479, filed on Apr. 24, 2023. The disclosure of that application is incorporated herein by reference.

### BACKGROUND

### Field

This disclosure relates to an apparatus and method for detecting and optionally determining the amount of bacteria in a fluid sample, wherein the sample is obtained from a human, non-human animal, or environmental source. In particular, the present disclosure relates to an apparatus that eliminates non-bacterial adenosine triphosphate (ATP) from a fluid sample in a first chamber and detects the presence of bacterial ATP in a second chamber. More particularly, the disclosure relates to an apparatus and method where the first and second chambers form a disposable cartridge and where the presence or amount of bacterial ATP is detected by emission of radiation, such as a light signal. The cartridge may be inserted in a device for performing detection automatically.

Joint replacement is a life-enhancing procedure for millions of people worldwide each year. Successful joint replacement provides pain relief, restores function and independence, and improves a patient's quality of life. While already a frequently performed procedure, the incidence of prosthesis implantation is expected to continue to rise. In the United States alone, there were 332,000 total hip and 719,000 total knee arthroplasties performed in 2010. These numbers are projected to increase substantially, reaching 850,000 in 2030 and 1,429,000 in 2040 for primary total hip arthroplasty (THA) and 1,921,000 in 2030 and 3,416,000 in 2040 for primary total knee arthroplasty (TKA) in the US alone. In concert with this trend in hip and knee arthroplasty there are also significant numbers of patients with prostheses of the shoulder, elbow and ankle.

Prosthetic joint infection (PJI) and septic arthritis (SA) are significant complications following total joint arthroplasty. PJI has an impact on patients, healthcare delivery institutions and society as a whole. It is challenging for the physician to manage, physically and is mentally disastrous for the patient. Moreover, the cost to the health care system and society as a whole is high.

The incidence of PJI varies with the joint involved. Reported incidences following total knee arthroplasty, total hip arthroplasty and total shoulder arthroplasty have been reported to be 0.25% to 2%, 0.5% to 1% and less than 1% respectively. Between 23-25% of revision TKA procedures and 12-15% revision THA procedures are performed for PJI. While the incidence is reported as low there is some evidence that suggests that the full scale of the issue is not reflected in the data. For example, cases managed with long term antibiotic suppression may not be captured in registry figures. Regardless of this, the number of patients is likely to increase over time as the number of arthroplasty procedures performed annually increases as outlined above. The cost of treating PJI is significant. It is estimated that in the US alone the projected spend on the treatment of PJI is $1.62 billion.

The diagnosis of PJI is based upon a combination of clinical findings, laboratory results from peripheral blood and synovial fluid, microbiological data, histological evaluation of periprosthetic tissue, intraoperative inspection, and, in some cases, radiographic results. There is no one test or finding that is 100% accurate for PJI diagnosis.

The general approach to PJI diagnosis is 2-fold. First, the question as to whether or not the joint is infected must be answered; second, if PJI is present, the causative microorganism(s) must be identified, and, in most cases, its antimicrobial susceptibility must be determined. Test performance may vary with joint type and also with timing post-arthroplasty implantation.

Diagnosing bacterial infections, including PJI, is currently performed using cell culture testing, which is considered the gold standard for diagnosis. The time required (minimum of 24-96 hours and sometimes up to two weeks) to obtain results may not be practical when the time to diagnosis and surgical intervention is critical. It is well established that the sooner surgical intervention or medical intervention can be performed, the better the outcome for patients with PJI.

In addition to potential delays in treatment, in up to 30% of bacterial cultures, bacteria is unable to be cultured and identified using traditional petri dish methods despite obvious secondary signs of infection. Currently, after a joint is aspirated, the aspirate is sent to a laboratory for gram stain and a culture and sensitivity test. The gram stain can detect white blood cells (WBC) s as well as bacteria but very frequently a varied number of WBCs are seen but no bacteria are present even from a patient that is infected. In 20-30% of those cases, cultures are read as negative, even though there is 100% certainty of an infection. In some disorders such as gout, pseudogout, or metallosis, the WBCs are elevated although there is no infection, which can dramatically alter treatment options. In addition, gram stain may not detect that live bacteria are present. Thus, pseudogout, gout, and a rheumatoid flair-up, as well as several other conditions that can look like infection but are not. Here, treatment is completely different from that of infection.

Another method for detecting bacteria in fluid from a suspected PJI is to identify biomarkers, such as of alpha-Defensins, in synovial fluids, for example, using enzyme-linked immunosorbent assay (ELISA), which is commercially available under the name Synovasure (Zimmer, Indiana, USA). Another method is to amplify suspected bacterial DNA using polymerase chain reaction (PCR) and template bacterial DNA to detect bacterial genetic material in the fluid of interest. Another method is to use gram staining to WBCs in samples aspirated from a potentially infected joint. These methods suffer from similar problems; they are often expensive and/or require long durations to be processed by laboratories of 24-48 hours (about 2 days) and sometimes 1-2 weeks. As a result, treatment of PJI is often delayed, and infections worsen until diagnosis is confirmed, negatively impacting clinical outcomes. Most dangerous is the possibility of a patient going into septic shock during the delay.

Known methods of detection that rely on using culture media or DNA amplification templates targeted for particular strains of bacteria may fail to detect other species or may fail to detect sessile, biofilm-embedded bacteria. The majority of PJIs are initiated through inadvertent introduction of normal microbial flora at the time of surgery. Early PJI (i.e., within three months after arthroplasty) mainly results from infection with virulent organisms, whereas delayed (i.e., 3-12 months after arthroplasty) or late (>12 months after arthroplasty) PJIs tend to be associated with low-virulence bacteria, such as Staphylococcus epidermidis. Infection by S. epidermidis may create a biofilm. Biofilm-related infections are difficult to diagnose, as traditional microbiological tests are optimized to detect free-floating but not sessile bacteria. Thus, known methods for detecting infection may fail to identify infected samples or may result in a misleading diagnosis.

Rapid and effective treatment of a PJI, particularly in THR and TKA, is critical to patient health and well-being. If infected, the patient must be placed on antibiotics. For an individual patient rapid management may mean a simple course of treatment; whereas, a delay gives a much higher chance of a considerably more difficult course of surgery, longer hospitalization, additional future surgeries that lead to a poorer end result.

Diagnosis and rapid treatment of infection is critical to ensure the best results for the patient. In certain cases, hours from diagnosis can be critical as native joint infections are considered surgical emergencies in order to preserve joint cartilage. Further, proper selections of antibiotics based on culture data is critical and delays in receiving information from the lab can exceed 3-5 days in order to adjust antibiotics to specific organisms.

U.S. Publication No. US 2022/0356505 A1 to Transformative Technologies provides for methods for detecting and/or determining bacterial in a liquid sample. The disclosure of that publication and any other publication cited herein are incorporated by reference in their entirety.

Thus, there is a need for an apparatus and method to detect and optionally determine the amounts of bacteria present in a fluid sample, wherein the sample is obtained from a source such as, for example, a human, a non-human animal (e.g. companion animals (e.g., cats, dogs, or rabbits), animals used in husbandry (e.g., cattle, sheep, goats, pigs, and poultry) or equines), or an environment whereby a technician can get reliable information regarding the presence and amount of bacteria in the sample within a few minutes after it is obtained.

### SUMMARY

The present disclosure relates to an apparatus to rapidly detect the presence of bacteria in a fluid sample. Such a sample can be a biological sample from a human or a non-human animal including, but not limited to blood, plasma, serum, milk or other lactation products, amniotic fluids, sputum, saliva, urine, semen, cerebrospinal fluid, uterine fluids, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof. The biological sample can be an environmental sample, including but not limited to a fluid or specimen obtained or derived from food products, food produce, poultry, meat, fish, beverages, dairy product, eggs, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, prisons, animal colony facilities, or any combinations thereof.

According to one non-limiting example, the biological sample is an aspirate from a patient's joint that is suspected of being infected. Also disclosed is a method whereby bacteria can be detected in a sample at a convenient location, such as at a patient's bedside or in a physician's office. According to one aspect, the disclosure includes a device with the capability to detect bacteria in synovial fluid rapidly, with high sensitivity/specificity, and that is inexpensive. Such a device may be used in a physician's office or clinic, a hospital emergency department, a surgical floor, an operating room or the like.

According to another non-limiting example, material to be tested is obtained by contacting a surface with a collection device, such as a swab, to gather particles, for example, dust, dander, or hair from the surface and immersing the collection device in a fluid to extract materials from the collection device to create a fluid sample.

In addition, biological samples may be tested directly after being sampled, but the disclosure is not limited to testing such fluids without further preparation. The disclosure includes apparatus and methods for testing fluids that have first been subject to other processing, such as by filtering, centrifugation, treatment with antibiotics, surfactants, buffers, enzymes, or other compounds, and by dilution, and other techniques known to those of skill in the field of the disclosure. For example, in situations where the biological sample is obtained using a swab or similar device, such as what is used to obtain samples from environment sources, the fluid sample is prepared by dissolving of mixing the biological sample in a solvent, such as a buffer (e.g., an EDTA buffer, a TRIS buffer, or a phosphate buffer) or saline.

According to one embodiment, a device for the rapid detection of bacteria in samples of fluid optically detects bacterial-generated ATP using luciferin/luciferase as the light-generating reagent. The device includes apparatus for destroying ATP from non-bacterial sources, such as somatic cells and endogenous ATP prior to detecting potential bacteria-generated ATP. According to one embodiment, the device provides a response (binary or quantitative) whether bacteria is present. According to one embodiment, such a device provides an output indicating a possible infection when the optical signal is indicative of any bacterial ATP. According to another embodiment, the device provides an output that there is a possible infection when the optical signal is indicative of an amount of bacterial ATP that exceeds a selected threshold. According to a further embodiment, the device is equipped with different bacteria specific markers and can provide a signal identifying specific bacteria or a specific category of bacteria to assist with proper antibiotic administration.

According to one embodiment, a biological sample is taken from a joint or other area of a patient's body that is suspected of being infected and placed in a first stage processing chamber. The processing chamber holds beads with ATP-reactive enzymes bound to the bead surfaces. According to one embodiment, an ATP-reactive enzyme is bound to the bead surfaces using techniques known to those of skill in the field of the invention, for example, using techniques described in P. Nyren, J. Biolumin Chemilumin. “Apyrase Immobilized on Paramagnetic Beads used to Improve Detection Limits in Bioluminometric ATP Monitoring,” 9 (1), pp. 29-34, doi: 10.1002/bio. 1170090106 (1994) or Fu et al., Peptide-Modified Surfaces for Enzyme Immobilization, PloS One, Apr. 1, 2011, Vol. 6, e18692. According to one embodiment, the ATP-reactive enzyme includes one or more of ATPase (e.g., apyrase), alkaline phosphatase, acidic phosphatase, hexokinase, adenosine phosphate deaminase and luciferase. The processing chamber also includes a somatic cell lysis agent, which may be provided as a powder or tablet with the processing chamber or coated onto the bead surfaces or other surfaces within the processing chamber.

According to one embodiment, the somatic cell lysis agent is a non-ionic surfactant or detergent selected to disrupt the membrane of selected cells. According to one embodiment, the somatic lysis agent is a non-ionic surfactant selected to selectively disrupt somatic cells but to leave bacterial cells, which may have more robust cell membranes, intact. According to one embodiment, the non-ionic surfactant is selected from the group consisting of Neonol AF9-10 (Nonoxynol-9), a saponin, an amphipathic glycoside, Triton X-100 (polyethylene glycol tert-octylphenyl ether), Lubrol (polyethylene glycolmonoacetyl ether), and combinations thereof.

When the sample is placed in the processing chamber, the somatic lysis agent dissolves into the sample and disrupts non-bacterial cells, releasing their cell contents. The ATPase or other ATP-reactive enzyme bound to the beads reacts with the ATP released from the somatic cells as well as any free-floating endogenous ATP, destroying ATP by converting it to ADP, AMP, or another compound. As a result, all or almost all free-floating ATP in the sample is destroyed. Any ATP remaining in the sample is exclusively contained within cells with relatively more robust cell walls that were not disrupted by the somatic lysis agent, such as bacteria. According to one embodiment, the reaction with the bead-bound ATPase is allowed to proceed a sufficient time to assure that all free-floating ATP is eliminated.

The processing chamber may be provided with a stirring mechanism. According to one embodiment, the chamber is connected with a mechanical vortexer. According to a further embodiment, the vortexer is connected with a controller to selectively energize the vortexer. According to another embodiment, a cartridge formed by the processing and detection chambers is in contact with an ultrasonic transducer. The transducer is connected with and controlled by a controller to mix the sample by sonification.

According to one embodiment, the beads are magnetic beads that include a paramagnetic or ferromagnetic component so that they can be manipulated by a magnetic field. One or more electromagnetic coils are positioned near the processing chamber to create a magnetic field within the chamber to move the beads or to fix the beads within the chamber. The coils may be connected with and controlled by the controller to modulate the magnetic field to selectively move the beads. According to one embodiment, the controller moves the beads to stir the sample to facilitate chemical reactions within the chamber. According to another embodiment, the processing chamber is vortexed or sonicated in addition to being stirred using a magnetic field to displace the beads. According to another embodiment, the controller energizes the coils to hold the beads in place within the processing chamber.

According to another embodiment, a light-emitting reagent, such as a luciferin/luciferase reagent is preloaded in the processing chamber. According to one embodiment, the luciferin/luciferase reagent is selected from the group consisting of the ATP Biomass Kit #CCK4 manufactured by Hygiena, the Bright Glo system manufactured by Promega, and any formulation which contains naturally occurring or genetically recombinant-luciferase and a suitable substrate, including luciferin.

According to one embodiment, freeze-dried luciferin/luciferase reagent is loaded as a powder, a compressed tablet, or is coated on an inner surface of the processing chamber. The luciferin/luciferase reagent dissolves in the sample and generates photons when reacted with ATP. According to another embodiment, the ATP-reactive enzymes bound to the bead surfaces include luciferase along with an ATPase such as apyrase, or other ATP-reactive enzymes. A suitable substrate for reacting with ATP and luciferase, such as luciferin, is provided in the processing chamber. ATP released from somatic cells disrupted by the somatic lysis agent reacts with the luciferin/luciferase reagent, emitting light.

The processing chamber is transparent or includes a transparent window through which light emitted by reaction of ATP can be transmitted. Adjacent to the processing chamber is a processing light sensor, such as a photon counter, avalanche diode, photocell, charged coupled device, photomultiplier tube, luminometer, or the like to detect light emitted by reaction of free-floating ATP in the sample.

According to one embodiment the intensity of the light diminishes as fee-floating ATP is consumed by the ATPase and the luciferin/luciferase reagent. A controller connected with the processing light sensor monitors the intensity of light emitted by reaction of free-floating ATP with the luciferin/luciferase and determines when the amount of light emitted has fallen below a threshold, indicating that all or almost all of the ATP remaining in solution has been destroyed. To assure that all free-floating ATP has been reacted, the controller may energize the electromagnetic coils to move the beads to stir the sample based on the detected light signal and/or may energize a vortexer or ultrasonic transducer connected with the processing chamber to stir the sample. By detecting an end point for the reaction of somatic and endogenous ATP, the controller can confirm that all or substantially all such ATP has been destroyed. According to one embodiment, the controller energizes a stirring mechanism in response to the light signal to confirm that ATP-destroying reactions have reached an end point.

According to one embodiment, once the somatically generated ATP has been destroyed, the controller energizes the electromagnet to hold the beads in place within the processing chamber. According to an alternative embodiment, a filter, a constriction, or other mechanism is provided to hold the beads within the processing chamber when the sample is transferred. Because the ATP reactive enzymes (e.g., ATPase) are bound to the beads, this contains that ATP-reactive enzymes within the confines of the processing chamber. By sequestering these ATP-reactive enzymes within the processing chamber, this prevents or reduces degradation of bacterial ATP in later steps.

A detection chamber is connected with the processing chamber. According to one embodiment, an opening, a tube, or another fluid connection is provided between the processing and detection chambers. According to one aspect, a mechanical actuator, such as a piston or pump, is provided to move the sample from the processing chamber into the detection chamber. According to another aspect, a valve is provided between the chambers to selectively allow the sample to move into the detection chamber. The mechanical actuator and/or valve are connected with and controlled by the controller.

According to one embodiment, the detection chamber includes a bacterial lysis agent, such as a second detergent surfactant, antibiotic or enzyme selected to disrupt bacterial cell membranes. The bacterial lysis agent may be selected from a group consisting of a bacteriophage lytic enzyme (endolysin), such as Lysostaphin, LysK, Lyse5h, LambdaSa2, OSH3b, KSN383, LysA, LysA2, Lysga Y, truncated lambda Sa2, H5CHAP-Lyso, Lysolv123-H5CHAP-OSH3b, or Plyc or may be a modified lytic enzyme (genetic or chimeric), a quaternary amine, an anionic, cationic, zwitterionic and/or nonionic surfactant, and combinations thereof.

Anionic surfactants may be selected from a group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauryl sarcosinate, perfluorononanoate, and perfluorooctanate (PFOA or PFO).

Cationic surfactants can be chosen from a group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

The zwitterionic surfactant may be sulfobetaine-3-10.

In one embodiment, the detection chamber holds light-generating reagent, such as luciferin/luciferase reagent, that reacts with free-floating ATP in the detection chamber. According to one embodiment, the bacterial lysis agent and luciferin/luciferase reagent are provided in dried form and provided as a powder or compressed into disks or tablets and provided within the detection chamber. In an alternative embodiment the light-generating reagent is present in both the processing and detection chambers.

According to other embodiments, a selective disrupting agent is provided in the processing and/or detection chamber that selectively disrupts membranes of certain types of bacteria, while leaving other types of bacteria intact. Such bacteria destroying agents include antibiotics. According to one embodiment, the selective disrupting agent is an antibiotic known to selectively destroy gram-positive bacteria, selected from the group consisting of one or more of methicillin, penicillin, cloxacillin, amoxicillin, erythromycin, and combinations thereof.

According to one embodiment, the gram-positive bacteria are selected from the group consisting of Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp., and Listeria spp.

According to another embodiment, the selective disrupting agent is an antibiotic known to destroy gram-negative bacteria, selected from the group consisting of an aminoglycoside, including gentamicin, amikacin, tobramycin, neomycin, and streptomycin, and combinations thereof.

According to one embodiment, the gram-positive bacteria are selected from the group consisting of Escherichia coli, Klebsiella pneumoniae, Pseudomonas aeruginosa, and Proteus mirabilis.

Bacteria disrupted by the selective disrupting agent in the processing chamber release free-floating ATP that is destroyed by the ATP-reactive enzyme. As a result, bacteria remaining in the processing chamber are resistant to the selective disrupting agent. According to one embodiment, the selective disrupting agent includes methicillin used to treat staph infections, such as S. aureus. If the sample includes S. aureus that is not disrupted during processing, detection of bacterial ATP in the sample may indicate infection by methicillin-resistant Staphylococcus aureus (MRSA).

The detection chamber is formed from a transparent material or includes a transparent window to allow light emitted from the reaction of bacterial ATP and luciferin/luciferase to exit the chamber. A detection light sensor is positioned adjacent to the window of the detection chamber to detect light emitted by chemical reactions in the detection chamber. The controller is operatively connected with the detection light sensor to monitor reaction of the light-generating species. Because somatic and endogenous ATP was removed from the sample in the processing chamber, the only free-floating ATP available to react with the light-generating species in the detection chamber is the ATP released when the bacteria were lysed. Because the ATP-reactive enzymes in the processing chamber, such as an ATPase (e.g., apyrase) is bound to the beads and are thereby sequestered from the detection chamber, bacterial ATP release in the detection chamber will not be destroyed before reacting with the luciferin/luciferase.

According to one embodiment, in response to light detected by the detection light sensor, the controller generates a signal to alert medical personnel of the possibility of an infection. According to one embodiment, in response to a light signal detected by the detection light sensor, the controller provides a binary signal to medical personnel, such as by illuminating a display to show a positive (possible infection) or negative (no infection). According to another embodiment, the controller generates an output that shows a quantitative analysis of the amount of bacterial ATP detected.

According to a further embodiment, a selective disrupting agent, such as an agent that selectively disrupts gram-negative or gram-positive bacteria as described above, is provided in the detection chamber. ATP released by the selectively disrupted bacteria reacts with the luciferin/luciferase reagent to generate a light signal that is detected by the detection light sensor, indicating the presence of the selectively disrupted bacteria.

According to one embodiment, the detection chamber is connected with a stirring mechanism such as a mechanical vortexer or ultrasonic transducer. According to a further embodiment, the same vortexer or transducer is connected with both the processing chamber and the detection chamber. According to a further embodiment, within the detection chamber are magnetic beads and an electromagnet is provided adjacent to the chamber. The electromagnet is energized to move the beads within the detection chamber to facilitate reaction of bacterial ATP with the luciferin/luciferase.

According to one embodiment of the disclosure, the processing chamber and detection chambers are part of a unitary assembly that form a disposable test cartridge. The chambers are preloaded with magnetic beads and chemical species discussed above. The cartridge is shaped to be removably inserted into a testing device. The testing device includes sensors and other mechanisms discussed above. Once testing of a sample is complete, the cartridge is removed from the device and disposed of. A new cartridge is used for each sample tested. According to one embodiment, the testing device is a compact unit suitable for use in an operating room, in a clinic, physician's office, or at a patient's bedside.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a diagram showing an apparatus to detect bacteria in a sample according to an embodiment of the disclosure;

FIG. 2 shows a sample cartridge and testing device according to embodiments of the disclosure;

FIG. 3 shows a syringe for aspirating and processing a sample according to an embodiment of the disclosure; and

FIG. 4 is a diagram showing an apparatus to detect bacteria in a sample according to a further embodiment of the disclosure.

### DETAILED DESCRIPTION

Embodiments are described in terms of testing biological materials obtained from a human, such as a patient suffering from a disease. The disclosure is not limited to devices to diagnose or treat humans and is applicable to treatment and diagnose non-human animals. This includes the testing of biological materials such as blood, plasma, serum, uterine fluids, milk or other lactation products, amniotic fluids, sputum, saliva, urine, semen, cerebrospinal fluid, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

The disclosure is not limited to the testing of biological materials, and includes testing of environmental and industrial materials including, but not limited to a fluid or specimen obtained or derived from food products, food produce, milk, poultry, eggs, meat, fish, beverages, dairy products, water (including wastewater), ponds, rivers, reservoirs, swimming pools, soils, food processing and/or packaging plants, agricultural places, farms, hydrocultures (including hydroponic food farms, aquaculture facilities), fish hatcheries, pharmaceutical manufacturing plants, medical facilities, hospitals, nursing homes, rehabilitation facilities, prisons, animal colony facilities, or any combination thereof.

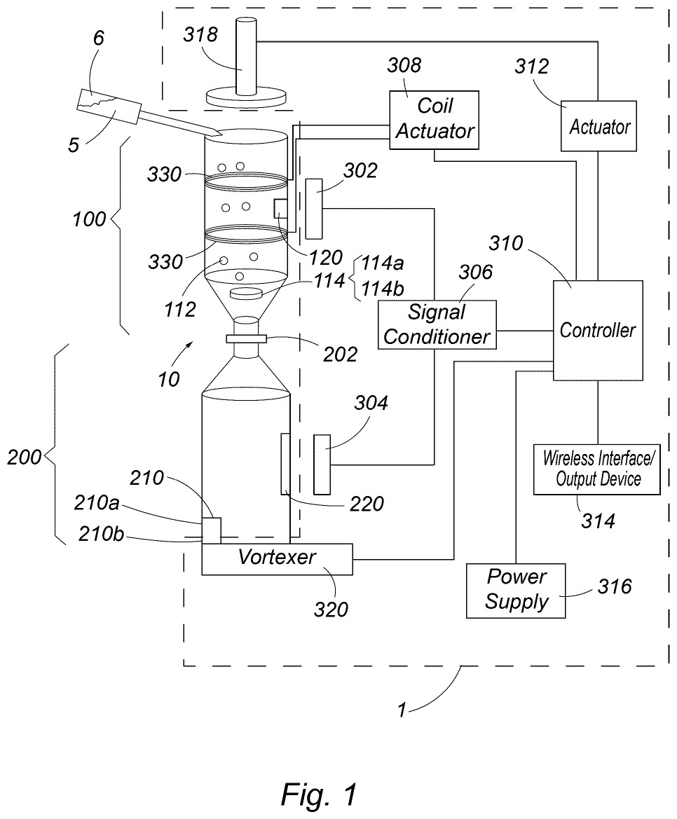

FIG. 1 shows an apparatus for detection of bacterial cells in sample 5 of a fluid according to an embodiment of the disclosure. Sample 5 may be an aspirate drawn from a body cavity suspected of being infected by bacteria using syringe 6 or may be obtained from another source. According to one embodiment, sample 5 is synovial fluid aspirated from a capsule surrounding a prosthetic joint replacement such as a TKA or THR.

Testing component 10 includes processing chamber 100 with an opening to allow sample 5 to be received. Within processing chamber 100 are a plurality of beads 112. According to one embodiment, beads 112 have one or more types of enzymes bound to their surfaces. According to one embodiment, the enzymes include ATP-disrupting enzymes that facilitate the conversion of ATP to another species, such as ADP. The enzymes may include, but are not limited to, adenosine triphosphatase (ATPase or apyrase), alkaline phosphatase, acidic phosphatase, hexokinase, adenosine phosphate deaminase, and luciferase.

Also provided in processing chamber 100 are one or more chemical reagents. According to one embodiment, the reagents are provided as dried powders compressed to form a solid mass, such as a tablet or disk 114. The chemical reagents include a somatic cell lysis agent 114a. Somatic lysis agent 114a may be a surfactant with a selected strength to preferentially disrupt somatic cells in sample 5. The disruptive strength of agent 114a is selected to preferentially disrupt somatic cells, but to leave bacterial cells, which generally have a more robust cell membrane, intact. According to one embodiment, somatic lysis agent 114a is selected from the group consisting of Neonol AF9-10 (Nonoxynol-9), a saponin, an amphipathic glycoside, Triton X-100 (polyethylene glycol tert-octylphenyl ether), Lubrol (polyethylene glycolmonoacetyl ether), and combinations thereof. According to other embodiments, somatic lysis agent 114a is selected from among reagents that preferentially disrupt somatic cells as known to those of ordinary skill in the field of the invention.

Processing chamber 100 is connected with detection chamber 200 by connection 202. According to one embodiment, connection 202 includes a valve to prevent sample 5 from flowing into detection chamber 200 until the valve is actuated. According to another embodiment, connection 202 is sized and has selected hydrodynamic properties to prevent sample 5 from flowing into detection chamber 200, until a pump mechanism, such as piston 318 is actuated, as will be described below, to force the sample through connection 202.

According to a further embodiment, connection 202 includes a filter or sufficiently narrow constriction to prevent passage of beads 112 (as well as any chemical species bound to the beads surfaces) from moving from processing chamber 100 to detection chamber 200.

In the case where sample 5 includes biological material such as from a human or non-human animal, sample 5 is expected to include somatic cells as well as endogenous ATP. Once it is delivered to processing chamber 100, sample 5 dissolves chemicals from disk 114, including somatic lysis agent 114a. Agent 114a preferentially disrupts somatic cells in sample 5, releasing the cell contents, including ATP and leaving bacteria cells in the sample intact. According to a further embodiment, processing chamber 100 also includes a selective disrupting agent selected to disrupt a selected type of bacteria, for example, gram-positive bacteria or gram-negative bacteria.

The ATP-disrupting enzyme bound to beads 112 converts the ATP released by the disrupted somatic cells, as well as endogenous ATP (and any bacterial ATP release by bacteria disrupted by the selective disruption agent), to ADP, AMP, or another compound. According to one embodiment, a mechanical mixing device such as mechanical vortexer or ultrasonic transducer 320 facilitates the reaction of sample 5 with chemical species in the processing chamber and the enzymes bound to beads 112. According to one embodiment, a sufficient time is allowed to assure that all, or almost all somatic cells have been disrupted and that all free-floating ATP in sample 5 has been converted to ADP. Vortexing or sonication may be applied to facilitate the destruction of all or almost all free-floating ATP.

Once all or almost all free-floating ATP has been destroyed, connection 202 is opened and/or piston 318 is actuated to move sample 5 from processing chamber 100 to detection chamber 200. Detection chamber 200 includes a bacterial cell lysis agent 210a. According to one embodiment agent 210a is a non-ionic surfactant or detergent, with sufficient strength to disrupt bacterial cells in sample 5. According to another embodiment, bacterial lysis agent 210a is selected from a group consisting of a bacteriophage lytic enzyme (endolysin), such as Lysostaphin, LysK, Lyse5h, LambdaSa2, OSH3b, KSN383, LysA, LysA2, LysgaY, truncated lambda Sa2, H5CHAP-Lyso, Lysolv123-H5CHAP-OSH3b, or Plyc or may be a modified lytic enzyme (genetic or chimeric), a quaternary amine, an anionic, cationic, zwitterionic and/or nonionic surfactant, and combinations thereof.

Anionic surfactants may be selected from a group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauryl sarcosinate, perfluorononanoate, and perfluorooctanate (PFOA or PFO).

Cationic surfactants can be chosen from a group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

The zwitterionic surfactant may be sulfobetaine-3-10.

Also included in detection chamber are compounds 210b that react with ATP to generate light. According to one embodiment, ATP-induced light generating compounds include a luciferin/luciferase reagent 210b. According to one embodiment, lysis agent 210a and light generating compounds 210b are compressed into a solid, such as one or more disks or tablets 210 within detection chamber 200. When sample 5 is delivered to detection chamber 200, lysis agent 210a and light-generating compounds 201b dissolve.

According to one embodiment, detection chamber 200 includes a transparent window 220. Because somatic and endogenous ATP was converted to ADP or another compound in the processing chamber, the only remaining ATP in sample 5 in detection chamber 200 is released by bacterial cells. A detection light sensor 304 is positioned to capture light emitted from reaction with the light generating compounds 210b. Detection of this light indicates the presence of bacteria in the sample. Light sensor 304 may be a photon counter, an avalanche diode, a photocell, a charge coupled device, a photomultiplier tube, a luminometer, or the like.

Vortexer or transducer 320 may be provided in connection with testing component 10, where motion by the vortexer stirs the contents of both chambers. According to another embodiment, two separate mechanical vortexers may be provided, with one vortexer designed to stir processing chamber 100 and one to stir detection chamber 200.

Detection light sensor 304 is coupled with a controller 310. According to one embodiment, a signal conditioner 306 is provided to process the detection signal from detection light sensor 304. According to one embodiment, individual photons liberated by the ATP/luciferin/luciferase reaction are converted to current or voltage signals by detection light sensor 304. Signal conditioner 306 may perform functions including filtering, amplification, integration, and/or baseline subtraction. According to one embodiment, signal conditioner 306 includes an analog-to-digital converter to provide a numerical output to controller 310 in proportion to an intensity or other parameter of the light detected by sensors 302 and 304. According to a further embodiment, signal conditioner 306 integrates signals generated by individual photons over time to provide a signal representative of the number of photons (that is, the number of ATP/luciferin/luciferase reactions) occurring over time to provide controller 310 with a slowly varying signal indicating a presence and/or concentration of bacterial ATP being reacted. According to one embodiment, one or more parameters of the signal correspond to the presence and/or amount of bacterially derived ATP. According to a further embodiment, that parameter is compared with a threshold value and, if the parameter is above the threshold, controller 310 reports a positive value (possible infection).

According to one embodiment, controller 310 is connected with one or more output devices 316, including, but not limited to indicator lights, a display monitor, an audio speaker, or a computer network-connected device. According to one embodiment, wireless interface 314 is connected with controller 310 to allow controller 310 to transmit information to a remote processer equipped with software to analyze the signal detected by detection light sensor 304. According to another embodiment, controller 310 is programmed with software to analyze the signal. Controller 310 is connected with power supply 316, such as a rechargeable battery or power cable.

According to a further embodiment of the disclosure, beads 112 in processing chamber 100 include a paramagnetic or ferromagnetic component so that the beads can be moved or held in place by a magnetic field. One or more electromagnetic coils 330 are provided near the processing chamber 100 so that, by applying current to one or more of the coils, beads 112 can be moved within the chamber. Coil actuator 308 provides current to coils 330 to generate time varying magnetic fields within chamber 100 to move beads 112 to stir sample 5. Coil actuator 308 can also generate a fixed magnetic field to hold the beads fixed within processing chamber 100. Actuator 308 is connected with and controlled by controller 310 to generate fixed or time-varying magnetic fields.

According to a further embodiment, processing chamber 100 is monitored by controller 310 to determine when somatic and endogenous ATP has been destroyed before sample 5 is transferred to detection chamber 200. Processing chamber 100 is formed by a transparent material or includes a transparent window 120. Processing light sensor 302 is positioned to detect light emitted by chemical reactions in processing chamber 100. The reagents in disk 114 include light-emitting compounds, for example, a luciferin/luciferase reagent 114b, which react with free-floating ATP and emit light from the reaction.

When sample 5 is placed in processing chamber 100, the luciferin/luciferase reagent 114b dissolves. As discussed above, somatic lysis agent 114a disrupts somatic cells release ATP, which reacts with ATPase on beads 112. Free-floating ATP also reacts with the luciferin/luciferase reagent in solution to generate a light signal. Sensor 302 detects the light emitted by reaction of free-floating ATP. According to one embodiment, an excess quantity of luciferin/luciferase reagent is provided so that only the concentration of free-floating ATP limits the rate of the light generating reaction so that the intensity of the light emitted by reaction of somatic and endogenous ATP is proportional to the concentration of ATP in solution. Sensor 302 is connected with controller 310 via signal conditioner 306.

Sample 5 is aspirated into syringe 6 and injected into processing chamber 100 dissolving lysis agent 114a and luciferin/luciferase reagent 114b. Controller 310 monitors the intensity of light emitted by luciferin/luciferase reactions in processing chamber 100. According to one embodiment, controller 310 is connected with and controls operation of vortexer 320 to stir sample 5 to facilitate the reaction. According to another embodiment, controller signals coil actuator 308 to energize coils 330 to move beads 112 to stir the sample. Controller 310 detects an endpoint for the removal of somatic and endogenous ATP based on the signal from sensor 302. According to one embodiment, controller 310 compares the signal with a threshold value and determines when the light intensity falls below the threshold indicating that all, or substantially all, free-floating ATP has been destroyed.

According to another embodiment, plunger 318 is adapted to fit within processing chamber 100 to displace sample 5 into the detection chamber 200. Plunger 318 is connected with plunger actuator 312. Controller 310 is connected with actuator 312 to cause sample 5 to be displaced. According to one embodiment, when controller 310 determines that the signal from sensor 302 is below a selected threshold, controller 310 actuates plunger 318 to move sample 5 into detection chamber 200. According to one embodiment, prior to actuating plunger 318, controller 310 energizes coils 330 to create a fixed magnetic field sufficient to hold beads 112 in processing chamber 100 and prevent the beads, as well as the ATP-reactive enzymes bound to the bead surfaces from moving into the detection chamber and destroying bacterially generated ATP.

Once sample 5 is in detection chamber 200, the sample dissolves the bacterial cell lysis agent 210a and light generating compounds 210b. If bacterial cells are present in sample 5, bacterial cell lysis agent 210a disrupts those cells, liberating bacterially derived ATP. The liberated ATP reacts with the light generating compounds 210b. If ATP is present in the sample, sensor 304 detects the signal. This signal indicates that bacteria were present and that the sample may have been collected from an infected body part, such as PJI.

According to one embodiment, bacterial cell lysis agent 210a includes a selective disrupting agent, such as an antibiotic that disrupts a selective type of bacteria, for example, gram-positive bacteria or gram-negative bacteria while leaving other types of bacteria intact. According to one embodiment, the selective disrupting agent is an antibiotic known to selectively destroy gram-positive bacteria, selected from the group consisting of one or more of methicillin, penicillin, cloxacillin, amoxicillin, erythromycin, and combinations thereof.

According to one embodiment, the gram-positive bacteria are selected from the group consisting of Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp., and Listeria spp.

According to another embodiment, the selective disrupting agent is an antibiotic known to destroy gram-negative bacteria, selected from the group consisting of an aminoglycoside, including gentamicin, amikacin, tobramycin, neomycin, and streptomycin, and combinations thereof.

According to one embodiment, the gram-negative bacteria are selected from the group consisting of Escherichia coli, Klebsiella pneumoniae, Pseudomonas aeruginosa, and Proteus mirabilis.

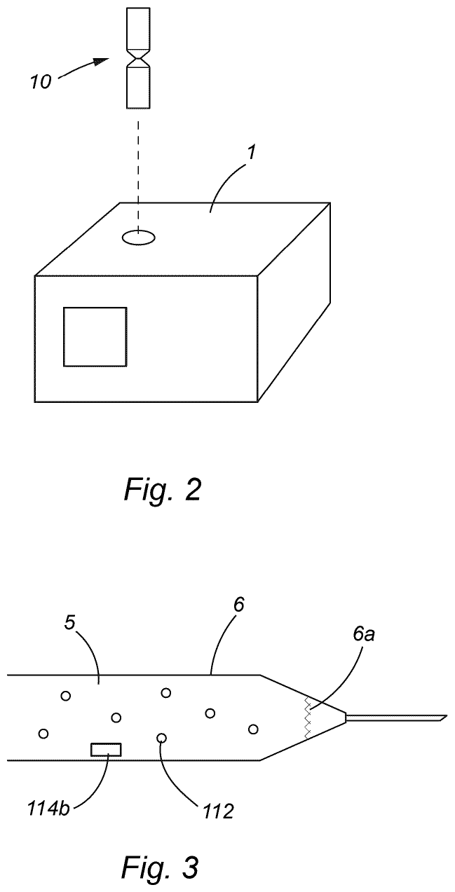

According to one embodiment, testing component 10 is a cartridge 10 that can be inserted into a testing device 1, as shown in FIG. 2. Device 1 houses controller 310, piston 318, sensors 302, 304, coils 330, vortexers 320, and related components. Device 1 includes an opening that accepts insertion of cartridge 10 to position in contact with vortexer 320 and with transparent windows 120, 220 adjacent to respective sensors 302, 304, as shown in FIG. 1.

According to one embodiment, sample 5 is provided in processing chamber 100 prior to insertion of cartridge 10 into device 1. Multiple samples 5 can be collected and stored in cartridges 10 and then tested one after another by inserting them in device 1.

FIG. 3 shows a syringe 6 according to another embodiment of the disclosure. Beads 112 with ATPase or other ATP destroying agent bound to their surfaces are preloaded in the syringe. Also preloaded in the syringe is somatic cell lysis agent 114b. Syringe 6 is used to aspirate a sample 5 of bodily fluid, such as fluid from a suspected PJI. As with the previous embodiment, somatic cells in the sample are disrupted by agent 114b to release somatic ATP and the reaction with ATPase destroys free-floating ATP. Filter 6a prevents beads 112 from passing out from syringe 6. Once somatic and endogenous ATD is destroyed, sample 5 is transferred to a detection chamber, such as detection chamber 200, as disclosed in previous embodiments.

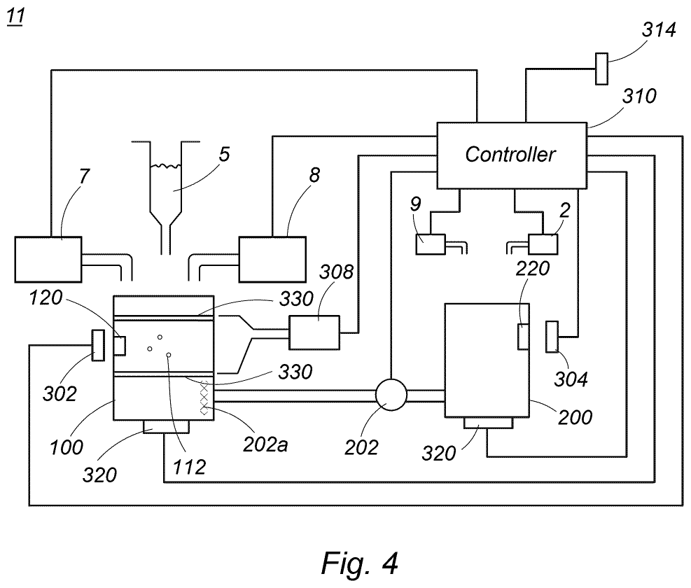

FIG. 4 shows a testing device 11 according to a further embodiment of the disclosure. Similar elements as discussed in previous embodiments are identified by the same element number. Device 11 includes a processing chamber 100 provided with a transparent window 120 adjacent a processing light sensor 302. Electromagnetic coils 330 are positioned adjacent to chamber 100. Mechanical vortexer 320 is coupled with chamber 100 to facilitate chemical reactions. One or more dispensers 7, 8 are connected with chamber 100. Dispensers 7, 8, coils 330, and vortexer 320 are connected with and controlled by controller 310.

In this embodiment, sample 5 is provided to chamber 100. Controller 310 causes dispenser 7 to add a selected amount of a somatic cell lysis agent such as agent 114b, as described in previous embodiments. to preferentially disrupt somatic cells to processing chamber 100. Vortexer 320 may be activated to facilitate disruption of somatic cells. According to one embodiment, beads 112, as described in previous embodiments with ATP-reactive enzymes bound to their surfaces are preloaded in chamber 100. Free-floating ATP reacts with the ATP-reactive enzymes, as with previous embodiments.

Coils 330 can be energized to move beads 112 to stir the sample. As with previous embodiments, a luciferin/luciferase reagent 114a may be provided in chamber 100, either preloaded, or added to the chamber by dispenser 8. Controller 310 receives signals from sensor 302 that indicate light-generating reactions showing the presence or concentration of ATP in chamber 100. When an end point is reached, controller 310 actuates pump 202 or other transfer mechanism to move the sample to detection chamber 200. Coils 330 are energized to create a magnetic field to hold the beads in chamber 100.

Detection chamber 200 may be preloaded with luciferin/luciferase reagent 210a and with a bacterial lysis agent 210b, or else dispenser 9 adds luciferin/luciferase to chamber 200 and dispenser 2 adds bacterial lysis agent to chamber 200 along with the sample. Chamber 200 includes transparent window 220. Detection light sensor 304 detects light emitted by reaction of ATP liberated from bacterial cells. Controller 310 monitors the signal from sensor 304 and, if light is detected, the controller 310 communicates that sample 5 may show infection, as described above.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. A device for detecting bacteria in a fluid sample comprising:
a processing chamber comprising,
a somatic cell lysis agent that selectively disrupts somatic cells to release somatic cell derived ATP-containing compounds into free-floating solution in the sample;
a plurality of beads; and
an ATP-reactive enzyme, wherein reaction between free-floating ATP-containing compounds in the sample and the ATP-reactive enzyme converts the ATP-containing compounds to corresponding AMP- or ADT-compounds, and wherein the ATP-reactive enzyme is bound to surfaces of the beads;
a detection chamber comprising,
a bacterial cell lysis agent; and
a light-generating reagent adapted to react with free-floating ATP to emit light;
a connection from the processing chamber to the detection chamber for selectively transferring the sample from the processing chamber to the detection chamber;
a bead retaining mechanism for retaining the beads in the processing chamber; and
a detection light sensor adapted to detect light emitted by the reaction of the light-generating species in the detection chamber.

2. The device of claim 1, wherein the fluid sample comprises biological material from a human or non-human animal.

3. The device of claim 2, wherein the fluid sample comprises biological material that is dissolved or mixed in a buffer or saline.

4. The device of claim 3, wherein the buffer is an EDTA buffer, a TRIS buffer, or a phosphate buffer.

5. The devices of claim 1, wherein the ATP-reactive enzyme is selected from the group consisting of ATPase, alkaline phosphatase, acidic phosphatase, hexokinase, adenosine phosphate deaminase and luciferase.

6. The device of claim 1, wherein the light-generating reagent is luciferin/luciferase.

7. The device of claim 1, further comprising a controller operatively connected with the detection light sensor, wherein the controller determines an amount of bacteria in the sample based on an output of the detection light sensor.

8. The device of claim 1, wherein the processing chamber further comprises an internal surface and wherein the internal surface is coated at least partially with luciferin/luciferase.

9. The device of claim 8, wherein the processing chamber further comprises a processing light sensor adapted to detect light emitted by reaction of the luciferin/luciferase with free-floating, somatic cell derived ATP in the processing chamber.

10. The device of claim 9, further comprising a controller connected with the processing light sensor and adapted to determine a progress of the conversion of free-floating ATP-containing compounds to corresponding AMP- or ADT-compounds by the ATP-reactive reagent in the processing chamber based on an output of the processing light sensor.

11. The device of claim 10, wherein the sample transfer mechanism is controlled by the controller, wherein the controller determines an end point for the reaction of free-floating ATP in the processing chamber with the ATP-reactive reagent based on the progress, and wherein the controller actuates the sample transfer mechanism to move the sample from the processing chamber to the detection chamber.

12. The device of claim 11, wherein the processing chamber further comprises one or more electromagnetic coils connected with the controller, wherein the plurality of beads each comprise a paramagnetic or ferromagnetic component, and wherein the controller is adapted to energize the coils to generate a magnetic field to manipulate the beads.

13. The device of claim 12, wherein the controller generates a time-varying magnetic field to move the beads within the processing chamber to mix the sample.

14. The device of claim 12, wherein the controller generates a fixed magnetic field to fix the beads within the processing chamber and forms the bead retaining mechanism.

15. The device of claim 1, wherein the bead retaining mechanism is a filter to allow passage of only objects smaller than the beads from the processing chamber to the detection chamber.

16. The device of claim 1, further comprising a mechanical vortexer or ultrasonic transducer connected with one or more of the processing chamber and the detection chamber, wherein the vortexer or transducer is connected with the controller and is selectively actuated by the controller.

17. The device of claim 1, wherein the somatic cell lysis agent is selected from a group consisting of Neonol AF9-10 (Nonoxynol-9), a saponin, an amphipathic glycoside, Triton X-100 (polyethylene glycol tert-octylphenyl ether), Lubrol (polyethylene glycolmonoacetyl ether), and combinations thereof.

18. The device of claim 1, wherein the bacterial cell lysis agent is selected from a group consisting of a bacteriophage lytic enzyme (endolysin), Lysostaphin, LysK, Lyse5h, LambdaSa2, OSH3b, KSN383, LysA, LysA2, LysgaY, truncated lambda Sa2, H5CHAP-Lyso, Lysolv123-H5CHAP-OSH3b, or Plyc or may be a modified lytic enzyme (genetic or chimeric), a quaternary amine, an anionic, cationic, zwitterionic and/or nonionic surfactant, and combinations thereof.

19. The device of claim 1, wherein the processing chamber comprises a syringe, wherein the bead retaining mechanism comprises a filter adapted to retain the beads within the syringe.

20. The device of claim 1, wherein the processing chamber and the detection chamber are formed as a single-use disposable cartridge, wherein the cartridge is inserted into a housing of the device to align the cartridge with the light sensor.
